(19) United States
(12) Patent Application Publication
(10) Pub. No.: US 2003/0103897 A1
(43) Pub. Date: Jun. 5, 2003

(54) Methods of using labelled verotoxin B subunit

(76) Inventor: Allan M. Green, Cambridge, MA (US)

Correspondence Address: LAHIVE & COCKFIELD, 28 STATE STREET, BOSTON, MA, 02109, US

(21) Appl. No.: 10/147,338

(22) Filed: May 15, 2002

Related U.S. Application Data
(60) Provisional application No. 60/291,137, filed on May 15, 2001.

(51) Int. Cl. A61K 51/00

(52) U.S. Cl. 424/1.41

(57) ABSTRACT

The invention pertains to methods for labeling antigen presenting cells, by contacting the antigen presenting cells with a labeled verotoxin. The invention also pertains to methods for determining the ability of an antigen presenting cell to stimulate an immune response.

### RELATED APPLICATIONS

This application claims priority to U.S. Provisional Application Serial No. 60/291,137, entitled “Methods of Using Labelled Verotoxin B Subunit,” filed on May 15, 2001. The entire contents of the aforementioned patent application are hereby incorporated herein by reference in their entirety.

### BACKGROUND OF THE INVENTION

During the 1960s, it was discovered that the two major classes of immune responses are mediated by different classes of lymphocytes: T cells, which develop in the thymus, are responsible for cell-mediated immunity; and B cells which produce antibodies and, in mammals, develop in the adult bone marrow or the fetal liver. Lymphocytes develop from pluripotent hematopoietic stem cells, which give rise to all of the blood cells, including red blood cells, white blood cells, and platelets. These stem cells are located primarily in hematopoietic tissues, such as the liver in fetuses and the bone marrow in adults. T cells develop in the thymus from precursor cells that migrate in from the hematopoietic tissues via the blood. In mammals, B cells develop from stem cells in the hematopoietic tissues themselves. Because they are sites where lymphocytes develop from precursor cells, the thymus and the hematopoietic tissues are referred to as primary (central) lymphoid organs.

Most lymphocytes die soon after they develop in a primary lymphoid organ. Others, however, mature and migrate via the blood to the secondary (peripheral) lymphoid organs—mainly, the lymph nodes, spleen, and epithelium-associated lymphoid tissues found in the gastrointestinal tract, respiratory tract, and skin. It is chiefly in the secondary lymphoid organs that T cells and B cells react with foreign antigens.

T cells must be activated to proliferate and differentiate before it can kill an infected target cell or help a macrophage or B cell. The initial activation of a T cell usually occurs when it recognizes a foreign peptide bound to an MHC molecule on the surface of an appropriate target cell. For a helper T cell, the appropriate target is an antigen-presenting cell.

Antigen-presenting cells are derived from the bone marrow and comprise a heterogeneous set of cells, including interdigitating dendritic cells in lymphoid organs and Langerhans cells in skin, as well as the B cells and macrophages that will subsequently be the target of T cell help. Together with thymus epithelial cells, which have a special role in T cell development, and activated T cells in some mammals, these specialized antigen-presenting cells are the only cell types that normally express class II MHC molecules. In addition to class II MHC molecules, antigen-presenting cells also express a second cell-surface molecule, called B7, that plays a crucial part in activating T cells.

The majority of T and B cells continuously recirculate between the blood and the secondary lymphoid tissues. In a lymph node, for example, lymphocytes leave the bloodstream, squeezing out between specialized endothelial cells; after percolating through the node, they accumulate in small lymphatic vessels that leave the node and connect with other lymphatic vessels, which then pass through other lymph nodes downstream. Passing into larger and larger vessels, the lymphocytes eventually enter the main lymphatic vessel (the thoracic duct), which carries them back into the blood. This continuous recirculation not only ensures that the appropriate lymphocytes will come into contact with antigen, it also ensures that appropriate lymphocytes encounter one another.

Lymphocyte recirculation depends on specific interactions between the lymphocyte cell surface and the surface of specialized endothelial cells lining small veins in the secondary lymphoid organs; because their endothelial cells are unusually tall, they are called postcapillary high endothelial venules. Many cell types in the blood come into contact with these high endothelial cells, but only lymphocytes adhere and then migrate out of the bloodstream. Different subpopulations of lymphocytes migrate through different lymphoid tissues, whereas most lymphocytes migrate into lymph nodes, for example, some migrate preferentially into Peyer's patches in the small intestine and constitute, in effect, a gut-specific subsystem of lymphocytes specialized for responding to antigens that enter the body from the intestine.

### SUMMARY OF THE INVENTION

In an embodiment, the invention pertains, at least in part, to a method for labelling a leukocyte, e.g., a white blood cell, e.g., an antigen presenting cell. The method includes contacting the leukocyte with a labeled verotoxin, such that the leukocyte is labeled.

The invention also includes methods for tracking a verotoxin binding cell in a subject, by contacting said verotoxin binding cell with a labeled verotoxin, administering said verotoxin binding cell to a subject, and detecting the label in the subject.

In another embodiment, the invention pertains to methods for determining the ability of a antigen presenting cell to stimulate a T-cell response to an antigenic agent in a subject. The method includes the steps of contacting the antigen presenting cell with a labeled verotoxin, contacting said antigen presenting cell with an antigenic agent, administering said antigen presenting cell to the subject, and detecting the location of the antigen presenting cell in the subject.

### DETAILED DESCRIPTION OF THE INVENTION

The invention pertains to a method for tracking antigen presenting cells in an organism.

The invention pertains, at least in part to a method for labelling a leukocyte, e.g., a white blood cell, e.g., an antigen presenting cell (APC). The method includes contacting the leukocyte with a labeled verotoxin, such that the leukocyte is labeled.

The term “verotoxin” includes verotoxin, subunits (e.g., B subunit), fragments (e.g., carbohydrate binding domain, Gb₃ binding domain, etc.), and mutants thereof which are capable of performing the intended function of the labeled verotoxin, e.g., label an antigen presenting cell. Subunits, fragments and mutants of shiga toxin and shiga-like toxin are also included as “verotoxin.”

The term “labeled verotoxin” includes verotoxin which has been labeled, such that it can be detected, e.g., by radioscintigraphy, X-ray, NMR, or other methods known in the art. Examples of labels which can be used to label verotoxin include, for example, ¹⁸F, ¹¹C, ¹³N, ⁴³K, ⁵²Cr, ⁵²Fe, ⁵⁵Mn, ⁵⁶Fe, ⁵⁷Co, ⁶⁷Cu, ⁶⁷Ga, ⁶⁸Ga, ⁷²As, ⁷⁵Br, ⁷⁶Br, ⁷⁷Br, ⁸⁰Br, ⁸¹Rb, ⁸¹Kr, ⁸²Br, ⁸⁷Sr, ⁸⁹Zr, ⁹⁰Y, ⁹⁷Ru, ⁹⁹Tc, ¹¹¹In, ¹¹³In, ¹²²I, ¹²³I, ¹²⁴I, ¹²⁵I, ¹²⁶I, ¹²⁷Cs, ¹²⁹Cs, ¹³¹I, ¹³²I, ¹⁵⁷Gd, ¹⁶²Dy, ¹⁹⁷Hg, ²⁰¹Tl, ²⁰³Pb, ²⁰⁶Bi, paramagnetic isotopes, x-ray labels, etc. Other labels which can be used include small molecules, such as biotin. In another embodiment, the verotoxin is labeled with a label that emit radiation at visual, IR, or near IR wavelengths. Examples of fluorescent labels include, but are not limited to, fluorescien, lissamine, phycoerythrin, rhodamine, Cy2, Cy3, Cy3.5, Cy5, Cy5.5, Cy7, FluorX, or others known in the art, such as those descibed in Haugland, Handbook of Fluorescent Probes and Research Chemicals, Molecular Probes, Inc.: Eugene, Oreg. (1996), incorporated herein by reference) Those of ordinary skill in the art will know of other suitable labels for binding to verotoxin, or will be able to ascertain such, using routine experimentation. Furthermore, the attachment of these labels to the verotoxin can be done using standard techniques common to those of ordinary skill in the art. In an embodiment, the amount of radiolabelled verotoxin administered to the subject is from about 0.1 to about 100 millicuries, from about 1 to about 10 millicuries, or, from about 2 to about 5 millicuries. In a further embodiment, the amount of a radiolabelled verotoxin administered to the subject is not fatal to the subject. In another embodiment, the amount does not exceed established radioactivity limits. In a further embodiment, the label absorb and emits energy at a wavelength which is distinguishable over the biological background of the cells.

In a further embodiment, the label may be attached by attached through using a linking group. The linking group may be attached covalently to the verotoxin and covalently or ionically to the label. For example, hydrazino nicotinamide (HYNIC) can be covalently attached to the verotoxin by reacting with amino groups and other basic functional groups of the verotoxin (e.g., lysine residues, terminal —NH₂ groups, etc.) and also be chelated to labels such as metal atoms. The process of labeling proteins such as verotoxin and verotoxin subunits is described in more detail in U.S. Pat. No. 5,206,370 and in Example 1.

The term “detect,” “detecting” or “detected” includes any methods known to one of skill in the art for locating a particular type of label in vivo. For diagnostic in vivo imaging, the type of detection instrument available is one factor in selecting a given label. The label chosen should have a type of signal which is detectable for a given type of instrument. In general, any conventional method for visualizing the labels in vivo can be utilized in accordance with this invention. In an embodiment, any method of scintigraphic imaging for diagnostic purposes can be utilized in accordance with this invention. In an embodiment, a radiolabeled verotoxin used for in vivo imaging will lack a particulate emission, but produce a large number of photons in a 140-200 keV range, which may be readily detected by conventional gamma cameras, gamma scanners, hand held gamma probe, etc (Makrigiorgos, et al., J.Nucl.Med., 31:1358-1363, 1990). Examples of such labels include, but are not limited to, ¹⁸F, ¹¹C, ¹³N, ⁴³K, ⁵²Cr, ⁵²Fe, ⁵⁵Mn, ⁵⁶Fe, ⁵⁷Co, ⁶⁷Cu, ⁶⁷Ga, ⁶⁸Ga, ⁷²As, ⁷⁵Br, ⁷⁶Br, ⁷⁷Br, ⁸⁰Br, ⁸¹Rb, ⁸¹Kr, ⁸²Br, ⁸⁷Sr, ⁸⁹Zr, ⁹⁰Y, ⁹⁷Ru, ⁹⁹Tc, ¹¹¹In, ¹¹³In, ¹²²I, ¹²³I, ¹²⁴I, ¹²⁵I, ¹²⁶I, ¹²⁷Cs, ¹²⁹Cs, ¹³¹I, ¹³²I, ¹⁵⁷Gd, ¹⁶²Dy, ¹⁹⁷Hg, ²⁰¹Tl, ²⁰³Pb, and ²⁰⁶Bi. To detect radioactivity provided by gamma emitter detectably labeled verotoxin, an instrument commonly known as a gamma camera (i.e., a system of scintillation crystals or photo multiplier tubes for analysis of radioactive decay) may be used to detect gamma emission from the detectably labeled verotoxin. To detect radioactivity provided by positron emitter detectably labeled verotoxin, techniques and instruments for positron emission tomography (PET) and single photon positron emission spectography (SPECT) are available to, and well-known in, the art. Those of ordinary skill in the art will also recognize that the labeled verotoxin of the invention may be coupled to paramagnetic isotopes for use in magnetic resonance imaging (MRI) (e.g., labels such as gadolinium (Gd)), may be coupled to paramagnetic isotopes for use in electron spin resonance (ESR) or may be covalently attached to contrast media for use in ultrasound. In general, any conventional method for detecting labels in vivo can be utilized.

The selection of the label for the labeled verotoxin and detection technique suitable for a given application is within the ordinary level of skill in the art. Factors to be considered in this respect include the existence of any host sensitivity to a particular radioisotope, in vivo toxicity and efficiency of such molecules, potential pharmaceutical interactions between the detectably labeled verotoxin and other medications taken by the host, the availability of particular detection instruments, and cost of materials.

For in vivo diagnostic imaging, the type of detection instrument available is one factor in selecting a given labeling agent. For radioactive labeling agents, the radioisotope chosen must have a type of decay which is detectable for a given type of instrument. In an embodiment, the half-life of the radioisotope label is long enough so that it is detectable at the time of maximum uptake by the target, but is short enough so that deleterious radiation with respect to the host is minimized. In a further embodiment, the radiolabel verotoxin used for in vivo imaging may lack a particle emission, but produce a large number of photons in the 140-250 keV range, which may be readily detected by conventional gamma cameras.

Typical examples of radiolabels which can be bound to the verotoxin of the invention include but are not limited to, ¹¹¹In, ⁹⁷Ru, ⁶⁷Ga, ⁶⁸Ga, ⁷²As, ⁸⁹Zr, ⁹⁰Y, ²⁰¹Tl, ¹⁵⁷Gd, ⁵⁵Mn, ¹⁶²Dy, ⁵²Cr, ⁵⁶Fe, ¹²²I, ¹²³I, ¹²⁴I, ¹²⁵I, ¹²⁶I, ¹³¹I, ⁸⁰Br, ⁸²Br, ⁷⁵Br, ⁷⁶Br, ⁷⁷Br, ⁸⁰Br, ¹⁸F, ¹¹C, ¹³N and ⁹⁹Tc. Those of ordinary skill in the art will be familiar with, or can readily ascertain, synthesis methods appropriate to the preparation of radioisotopically labeled verotoxin for use in the inventive method. For example, other suitable radioiodination labeling techniques are taught in Keough, et al, J. Labeled Compound Radiopharm., 14:83-90, 1978. In addition, techniques useful in labeling molecules with positron emitters (e.g., ¹⁸F) are known in the art and include the technique disclosed in Ishiwata, et al., Eur. J. Nucl. Med., 9:185-189, 1984 (¹⁸fluorine labeling of deoxyuridine). Techniques for labeling with non-halogen radioisotopes (such as ¹¹C) are also well-known and include the technique referred to in Kubota, et al., Jpn. J. Cancer Res., 80:778-782, 1989.

For in vitro use, there are many different labels and methods of labeling known to those of ordinary skill in the art. Examples of the types of labels which can be used in the present invention include enzymes, radioisotopes, fluorescent compounds, colloidal metals, chemiluminescent compounds, and bio-luminescent compounds. Another labeling technique which may result in greater sensitivity consists of coupling the antibodies to low molecular weight haptens. These haptens can then be specifically detected by means of a second reaction. For example, it is common to use haptens such as biotin, which reacts with avidin, or dinitrophenol, pyridoxal, or fluorescein, which can react with specific anti-hapten antibodies. Those of ordinary skill in the art will know of other suitable labels for binding to verotoxin, or will be able to ascertain such, using routine experimentation. Furthermore, the binding of these labels to the verotoxin can be done using standard techniques common to those of ordinary skill in the art.

The term “leukocytes” includes antigen presenting cells, other white blood cells, and cells from the myeloid and lymphoid cell lineages. Examples of leukocytes include, but are not limited to, lymphocytes (e.g. B lymphocytes, T lymphocytes, etc.), monocytes, neutrophils, eosinophils, macrophages, dendritic cells, and basophils.

“Antigen presenting cells” or “APCs” are cells that are capable of activating T cells, and include, but are not limited to, certain macrophages, B cells and dendritic cells. In a further embodiment, antigen presenting cells include verotoxin binding cells.

The term “dendritic cell” includes members of a diverse population of morphologically similar cell types found in lymphoid or non-lymphoid tissues. These cells are characterized by their distinctive morphology and high levels of surface MHC-class II expression (Steinman, et al., Ann. Rev. Immunol. 9: 271 (1991)). These cells can be isolated from a number of tissue sources and, for example, peripheral blood. The term includes Langerhans cells, interstitial dendritic cells, interdigitating dendritic cells, follicular dendritic cells and circulating dendritic cells. Langerhans cells are found in the epidermis and mucous membranes. Interstitial dendritic cells populate most organs such as the heart, lungs, liver, kidney, and gastrointestinal tract. Interdigiting dendritic cells are present in T-cell areas of the secondary lymphoid tissue and the thymic medulla. Circulating dendritic cells include “veiled cells” which constitute about 0.1% of the blood leukocytes.

In general, dendritic cells are covered with a maze of long membrane processes resembling dendrites of nerve cells. Due to their long dendritic processes, dendritic cells have been challenging to study using conventional procedures for isolating lymphocytes and accessory immune-system cells. Dendritic cells tend to express high levels of both class II MHC molecules and the co-stimulatory B7 molecule. For this reason, they are more potent antigen-presenting cells than macrophages and B cells, both of which need to be activated before they can function as APCs. After capturing an antigen in the tissues by phagocytosis or by endocytosis, dendritic cells migrate into the blood of lymph and circulate to various lymphoid organs where they present the antigen to T lymphocytes.

Follicular dendritic cells are also included. Follicular dendritic cells do not express class II MHC molecules and therefore do not function as antigen presenting cells for T_{H}-cell activation. They are located exclusively in the follicles of the lymph nodes and express high levels of membrane receptors for antibody and complement. Binding of circulating antibody-antigen complexes by these receptors is thought to facilitate B-cell activation in lymph nodes.

Dendritic cells may be used in tumor vaccines or for other immunological purposes (DiNicola, M. et al., Cytokines Mol. Ther. (1998) 4(4):266-273; Choi, D. et al., Clin Cancer Res. (1998) 4(11):2709-16; Soligo, D. et al. Br. J. Haematol. (1998) 101(2):352-63). Dendritic cells may be generated from hematopoietic progenitor cells or harvested from a patient by methods known to those skilled in the art. Dendritic cells can be used to present tumor or other antigens, or may be fused to the tumor cell making tumor antigens an intrinsic component of the dendritic cell surface.

The antigens presented by the dendritic or antigen presenting cells may be specific for, for example, breast, ovarian, testicular, prostate, lung, bowel, rectal, prostate, pancreatic, stomach, brain, or skin (e.g., melanoma) cancers.

The term “verotoxin binding cells” include cells with are capable of being labeled with a labeled verotoxin. Examples of verotoxin binding cells include cells which express Gb₃ (e.g., CD77), dendritic cells, hematopoietic progenitor cells, etc.

In a further embodiment, the invention pertains, at least in part to a method for labelling dendritic cells, by contacting said cells with a radiolabelled verotoxin B subunit.

In another embodiment, the invention also pertains to a method for tracking verotoxin binding cells in a subject. The invention includes contacting a verotoxin binding cell with a labeled verotoxin, administering said verotoxin binding cell to a subject, and detecting the label in the subject. The cell may be administered to the subject in a pharmaceutically acceptable carrier. Advantageously, the cells are administered parentally, e.g., intravenously or intranodally.

In a further embodiment, the labeled verotoxin is radiolabeled and may advantageously, comprise verotoxin B or fragment thereof, e.g., a fragment or mutant which is capable of exibiting verotoxin B binding activity. The cell may be exposed to a differentiation agent prior to administration to the subject.

The term “subject” includes organisms which have cells which can be labeled using labeled verotoxin. Examples of subjects include mammals such as, for example, rodents (e.g. rats, mice, hamsters, squirrels), horses, cows, pigs, sheep, cats, dogs, bears, goats, and primates (e.g., monkeys, chimpanzees, gorillas, and, preferably, humans).

In a further embodiment, the mammal may be suffering from an antigen associated state.

The term “antigen” includes agents which provoke an immune response independently and those which are provoke an immune response when incorporated in to a conjugate of the invention. The term “antigen epitope” includes fragments of proteins capable of determining antigenicity. An epitope may comprise, for example, a peptide of six to eight residues in length (Berzofsky, J. et al., (1993) in Paul, W., Ed., Fundamental Immunology, Raven Press, New York, p.246). Some epitope may be significantly larger. The affinity of an antibody molecule for its cognate epitope ranges from low, e.g. 10⁻⁶ M, to high, e.g., 10⁻¹¹ M.

For example, antigens include proteins and other molecules which are specifically associated with surfaces of particular types of cancer cells, e.g. tumor cells. Many forms of cancer can be characterized by production of proteins associated with that form of the disease, and are not found in normal tissue. Often these proteins are used at a specific stage of embryonic development, and are not observed during normal adult lifetime. These antigens are particularly useful as a source of epitopes for anticancer vaccines. Examples of tumor antigens that are envisioned as antigens for the conjugates of the present invention include those corresponding to cancers affecting the breast, ovarian, lung, skin, and brain. For example, breast tumors may be characterized by abnormally expressed receptors, e.g. those of the human-EGF-like receptor family (HER). Additionally, the nestin protein, which is expressed by neuroepithelial stem cells during normal mammalian fetal development, is also expressed on tumors of the central nervous system, including most forms of brain cancer (McKay, D. G. Ronald,U.S. Pat. No. 5,338,839). It is also expressed on melanomas found on the skin and on those which have metastasized to other tissues (V. A. Florenes, et al., 1994, Cancer Res. 54: 354-6). The present invention contemplates incorporating these antigens or epitopes of these antigens into compounds of the invention. Preferably, the antigens of the toxin-antigen conjugates of the invention are peptides associated with melanoma which may be derived, for example, recombinantly or from tumor cell lysate.

Other examples of tumors expressing antigens contemplated by the present invention include Wilm's tumor (A. J. Buckler, et al. U.S. Pat. No. 5,350,840), gastrointestinal cancer (R. Fishel et al, International Application WO 95/14085, May 26, 1995), cancers characterized by development of multiple drug resistance during chemotherapy (J. M. Croop et al., U.S. Pat. No. 5,198,344), and cancers characterized by the presence of at least one of a large number of oncogenes well known to the skilled artisan, such as Rb, ras, and c-myc, the sequences of which are available for analysis to those with skill in the art.

Other antigens include “tissue-specific tumor antigens” which include antigens which are common to specific tumor types. For example, malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD 19, CD20, idiotype) have been used as targets for passive immunotherapy with monoclonal antibodies with limited success.

Thus, examples of tissue-specific tumor antigens include, but are not limited to prostatic acid phosphatase (PAP; associated with prostatic tumors), Melan-A/MART-1 (associated with melanoma; Coulie et al., 1994, J. Exp. Med. 180:35, Hawakami et al., 1994, PNAS 91:3515, Bakker et al., 1994, J. Exp. Med. 179:1005), tyrosinase/albino (associated with melanoma; Kawakami et al., 1994, J. Exp. Med.), and CD 19, CD20 and CD37 (associated with lymphoma).

Likewise, oncogene product peptide antigens have been identified that are common to specific tumor types. These polypeptides will find use in the polypeptide complexes of the present invention as reagents that can be used generally to stimulate T-cell responses effective to react with tumors bearing such antigens. oncogene product peptide antigens include but are not limited to HER-2/neu (Beckmann et al., 1992, Eur. J. Cancer 28:322) associated with human breast and gynecological cancers, carcinoembryonic antigen (CEA) associated with cancer of the pancreas.

A variety of tumor markers are known in the art or are commercially available and include, but are not limited to the tissue-specific antigens that include cytokeratins, prostate-specific antigen, gp75/brown (Brichard et al., 1993, J. Exp. Med. 178:489) associated with melanoma, melanotransferrin (Real et al., 1984, J. Exp. Med. 160:1219), MUC1 (Barnd, 1989, PNAS USA 86:7159 and Vijayasaradhi et al., 1990, J. Exp. Med. 171:1375) associated with pancreas and breast cancer; oncogene/tumor suppressor genes that include EGF-R (Osborne et al., 1980), estrogen receptor, progesterone receptor, retinoblastoma gene product, myc associated with lung cancer, ras, p53, nonmutant associated with breast tumors, MAGE-1,3 (van der Bruggen et al., 1991, Science 254:1643 and Gaugler et al., 1994, J. Exp. Med. 179:921) associated with melanoma, lung, and other cancers.

Alternatively, antigens of the invention include or be associated with the surfaces or secretion products of micro-organisms or pathogens. The term “pathogen” is meant to include organisms that cause disorders, such disorders produced by one or more particular species of bacteria, viruses, fungi, and protozoans which are disease-producing organisms. Examples of pathogens include gram-negative bacterial species such as Escherichia coli serotype 0157:H7, Helicobacter pylori, H. mustelae, Haemophilus influenzae and H. ducreyi, Pseudomonas aeruginosa, Shigella dysenteria, Salmonella typhi and S. paratyphi; Gram-positive bacterial species such as Mycobacterium tuberculosis, M. leprae, Clostridium tetani, Staphylococcus aureus, and Streptococcus hemolyticus; obligate intracellular bacterial organisms such as Rickettsia and Chlamydia species; retroviruses, which are RNA containing viruses that use reverse transcriptase to synthesize complementary DNA, including but not limited to HIV-1, and -2 (e.g., antigens such as gp120, gp41, gag, RT, NEF, VIF, etc.); other pathogenic viruses such HSV-I and -II, non-A non-B non-C hepatitis virus, pox viruses, influenza (e.g., antigens such as HA, core, matrix, etc.), EBV (e.g., antigens such as EBNA, BFLF1, BOLF1, BGLF2, LMP2a, LMP2b, BBRF1, BBRF2, and P11L27), human papilloma virus and rabies viruses; fungi such as Candida and Aspergillus species; protozoa such as Cryptosporidium parvum, Entamoeba histolytica and Giardia lamblia; and animal pathogens such as Newcastle disease virus. Obtaining unique epitopes from these organisms by screening proteins and by assaying peptides in vitro are commonly known to those skilled in the art; many examples have been described and the appropriate amino acid residue sequence may be accessed from Genbank.

The term “infection” is meant to include persistence and growth of a pathogen in a subject host. While symptoms used to diagnose the presence of infection include fever, inflammation, pain, joint and muscular sensations at or near sites of infection, the absence of one or more of these symptoms do not preclude infection in a subject host organism. The term “inflammation” indicates a set of host reactions that accompany infection, and may also be present in the absence of infection, for example, as a symptom of autoimmune reactions, degenerative diseases, tissue remodeling disorders, exposure to allergens, and/or other conditions. Inflammatory responses include cellular processes such as neutrophil, mast cell and basophil degranulation with associated release of proteases, histamines, and superoxide generation, and production of and responses to cytokines such as interferons and tumor necrosis factor.

Antigens also may include allergens. An “allergen” refers to a substance that can induce an allergic or asthmatic response in a susceptible subject. The number of allergens that elicit a sensitive response in a proportion of a population is enormous, and includes pollens, insect venoms, animal dander, dust mite proteins, fungal spores and drugs (e.g. penicillin). Examples of natural animal and plant allergens include proteins specific to the following genera: Felis (Felis domesticus); Canis (Canis familiaris); Dermatophagoides (e.g. Dermatophagoides farinae); Periplaneta (e.g. Periplaneta americana); Ambrosia (Ambrosia artemiisfolia; Lolium (e.g. Lolium perenne or Lolium multiflorum); Cryptomeria (Cryptomeria japonica); Alternaria (Alternaria alternata); Alnus (Alnus gultinosa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis). An “allergen associated state” includes states which are the resulting from an allergic or asthmatic response to an allergen.

The term “antigen associated state” includes micro-organism or pathogenic infections, allergen associated states, virus associated states (e.g., HIV, EBV, etc.) and, preferably, tumors such as, for example, breast, ovarian, brain, skin, lung, etc. In an embodiment, the antigen-related state is melanoma.

The term “treating” includes preventing and curing as well as ameliorating at least one symptom of the antigen associated state. It also includes the initiation of an immune response against an antigen associated state that the mammal may be susceptible to, but not necessarily suffering from. For example, in a mammal at risk for melanoma, a conjugate of the invention may be administered to said mammal, thus generating an immune response so as to prevent or delay the initiation of the potential melanoma.

The term “dendritic cells” include Langerhans cells, interstitial dendritic cells, interdigitating dendritic cells, follicular dendritic cells and circulating dendritic cells. Langerhans cells are found in the epidermis and mucous membranes. Interstitial dendritic cells populate most organs such as the heart, lungs, liver, kidney, and gastrointestinal tract. Interdigiting dendritic cells are present in T-cell areas of the secondary lymphoid tissue and the thymic medulla. Circulating dendritic cells include “veiled cells” which constitute about 0.1% of the blood leukocytes.

In general, dendritic cells are covered with a maze of long membrane processes resembling dendrites of nerve cells. Due to their long dendritic processes, dendritic cells have been challenging to study using conventional procedures for isolating lymphocytes and accessory immune-system cells. Dendritic cells tend to express high levels of both class II MHC molecules and the co-stimulatory B7 molecule. For this reason, they are more potent antigen-presenting cells than macrophages and B cells, both of which need to be activated before they can function as APCs. After capturing an antigen in the tissues by phagocytosis or by endocytosis, dendritic cells migrate into the blood of lymph and circulate to various lymphoid organs where they present the antigen to T lymphocytes.

The term “administering” includes routes of administration which allow the labeled verotoxin or the labeled cells of the invention to perform their intended functions. Depending on the route of administration, the labeled verotoxin or the labeled cells of the invention can be coated with or disposed in a selected material to protect it from natural conditions which may detrimentally effect its ability to perform its intended function. The labeled verotoxin or the labeled cells of the invention can be administered alone or with a pharmaceutically acceptable carrier. Further, the labeled verotoxin or the labeled cells of the invention can be administered as a mixture, which also can be coadministered with a pharmaceutically acceptable carrier. The labeled verotoxin or the labeled cells of the invention can be administered prior to the onset of an antigen-related state, or after the onset of an antigen-related state.

In another embodiment, the invention features a composition which includes a labeled verotoxin or the labeled cells of the invention and a pharmaceutically acceptable carrier. The phrase “pharmaceutically acceptable carrier” means a pharmaceutically acceptable material, composition or vehicle, such as a liquid or solid filler, diluent, excipient, solvent or encapsulating material, involved in carrying or transporting the cells or labeled verotoxins of the present invention within or to the subject such that they can perform their respective intended functions. Typically, compositions are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Methods of preparing these formulations or compositions include the step of bringing into association a cell or a labeled verotoxin of the present invention with the carrier and, optionally, one or more accessory ingredients. In general, the formulations are prepared by uniformly and intimately bringing into association a cell or a labeled verotoxin of the present invention with liquid carriers.

Dosage forms for the topical or transdermal administration of a labeled verotoxin of this invention include powders, sprays, ointments, pastes, creams, lotions, gels, solutions, patches and inhalants. The labeled verotoxin may be mixed under sterile conditions with a pharmaceutically acceptable carrier, and with any preservatives, buffers, or propellants which may be required.

Transdermal patches have the added advantage of providing controlled delivery of a labeled verotoxin of the present invention to the body. Such dosage forms can be made by dissolving or dispersing the labeled verotoxin in the proper medium. Absorption enhancers can also be used to increase the flux of the labeled verotoxin across the skin. The rate of such flux can be controlled by either providing a rate controlling membrane or dispersing the labeled verotoxin in a polymer matrix or gel.

Pharmaceutical compositions of this invention suitable for parenteral administration comprise one or more cells or labeled verotoxins of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. Injectable depot forms are made by forming microencapsule matrices of the cells or labeled verotoxins in biodegradable polymers such as polylactide-polyglycolide. Depending on the ratio of cells or toxin to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, or topically. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” includes modes of administration other than enteral and topical administration, usually by injection, and includes, without limitation, intravenous, intramuscular, intraarterial, intrathecal, intracapsular, intranodally, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a cell or a labeled verotoxin of the invention other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In an embodiment, the invention pertains to methods for determining the ability of an antigen presenting cell to stimulate an immune response to an antigenic agent in a subject. The method includes contacting the antigen presenting cell with a labeled verotoxin and an antigenic agent, administering the cell to the subject, and detecting the location of the cell in said subject. Advantageously, the subject is a mammal, e.g., a human, e.g., a human suffering or at risk of suffering from an antigen-associated state. In an embodiment, the labeled verotoxin is radiolabelled (e.g., with ⁴³K, ⁵²Fe, ⁵⁷Co, ⁶⁷Cu, ⁶⁷Ga, ⁷⁷Br, ⁸¹Rb, ⁸¹Kr, ⁸⁷Sr, ⁹⁹Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb, or ²⁰⁶Bi). Examples of antigen presenting cells include dendritic cells and Langerhans cells.

The language “immune response” includes any response of the body against the antigen or antigenic epitope of the antigenic agent. The immune response includes the production of antibodies against the antigen or antigenic epitope, as well as any response by T cells to the antigenic agent or the antigen presenting cells, e.g., through entry into the class I pathway of antigen processing. The immune response may be ex vivo or in vivo.

The language “antigenic agent” include agents which comprise one or more antigens or antigenic epitopes which are capable of being expressed by an antigen presenting cell. The antigenic agent may comprise, for example, a toxin subunit (e.g., a verotoxin B subunit) and an antigen or antigenic isotope. In certain embodiments, the antigenic agent may be a labeled verotoxin B subunit liked to an antigen or antigenic epitope such that the labeled verotoxin and the antigenic agent are the same molecule. Examples of antigens include bacterial, viral, tumor (e.g., skin, brain, ovarian, or breast tumor antigens)or other antigens associated with diseases, as described above. Other antigenic agents include those described in U.S. Pat. No. 6,080,409.

For example, an antigenic agent may be induced in the class I pathway of a mature dendritic cell in vitro, by isolating antigen presenting cells (e.g., dendritic cells), “pulsing” or contacting them with the antigenic agent and labeled verotoxin (in combination or seperately) for an effective period of time, then using the pulsed antigen presenting cells to stimulate autologous T-cells in vitro or in vivo. In the latter case, an effective amount of the pulsed antigen presenting cells are administered to the subject. The response of the subject is measured by detecting the labeled verotoxin in the subject. Multiple doses may be administered to produce an adequate response.

Those skilled in the art will recognize, or be able to ascertain using no more than routine experimentation, many equivalents to the specific embodiments and methods described herein. Such equivalents are intended to be encompassed by the scope of the following claims. All patents, patent applications, and literature references cited herein are hereby expressly incorporated by reference.

### EXAMPLES

### Example 1

Conjugation of HYNIC to the B Subunit of Verotoxin

To a solution of 10 mg verotoxin B subunit in 2 ml 0.1M sodium phosphate buffer (pH 7.8) is added 17.2 μl of 30 mM hydrazino nicotinamide hydrochloride in dimethylformamide. After stirring for 5 hours at room temperature, the reaction mixture is dialyzed against 0.1M sodium acetate buffer (pH 5.2). The number of hydrazino groups conjugated onto the protein is measured by the method of T. P. King et al. (Biochemistry, 25:5774, 1986).

Labelling of Conjugated Verotoxin with ⁹⁹Tc

A DuPont Tc-glucoscan kit is reconstituted with 3 mL of water containing 10 mCi of ⁹⁹TcO₄⁻. 250 μl of this solution is mixed with 250 μl of 1-5 mg/ml conjugated verotoxin in 0.1M sodium acetate buffer (pH 5.2). The conjugated verotoxin is then incubated with the radiolabel for an hour. 800 uCi of Tc labelled verotoxin is injected intranodally into rats. At 24 hours, the rats are sacrificed and the distribution of radioactivity is measured.

1. A method for labeling a leukocyte, comprising:
contacting said leukocyte with a labeled verotoxin, such that said leukocyte is labeled.

2. The method of claim 1, wherein said leukocyte is an antigen presenting cell.

3. The method of claim 1, wherein said verotoxin comprises the B subunit of verotoxin.

4. The method of claim 1, wherein said verotoxin is labeled with a radiolabel.

5. The method of claim 4, wherein said radiolabel is selected from the group consisting of ⁴³K, ⁵²Fe, ⁵⁷Co, ⁶⁷Cu, ⁶⁷Ga, ⁷⁷Br, ⁸¹Rb, ⁸¹Kr, ⁸⁷Sr, ⁹⁹Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb, and ²⁰⁶Bi.

6. The method of claim 1, wherein said verotoxin is labeled with a fluorescent label.

7. The method of claim 6, wherein said fluorescent label is fluorescien, lissamine, phycoerythrin, rhodamine, Cy2, Cy3, Cy3.5, Cy5, Cy5.5, Cy7, or FluorX.

8. The method of claim 7, wherein said fluorescent label is Cy5.5.

9. The method of claim 2, wherein said antigen presenting cell is a dendritic cell.

10. A method for tracking a verotoxin binding cell in a subject comprising:
contacting said verotoxin binding cell with a labeled verotoxin;
administering said verotoxin binding cell to a subject; and
detecting said label in said subject,
thereby tracking said verotoxin binding cell in said subject.

11. The method of claim 10, wherein said verotoxin binding cell is radiolabeled.

12. The method of claim 11, wherein said radiolabel is chelated to hydrazino nicotinamide, which is covalently attached to said verotoxin.

13. The method of claim 11, wherein said radio label is selected from the group consisting of ⁴³K, ⁵²Fe, ⁵⁷Co, ⁶⁷Cu, ⁶⁷Ga, ⁷⁷Br, ⁸¹Rb, ⁸¹Kr, ⁸⁷Sr, ⁹⁹Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb, and ²⁰⁶Bi.

14. The method of claim 12, wherein said radiolabel is ⁹⁹Tc, ¹¹¹In, ¹³¹I, ¹²⁵I, ¹¹³In, ¹²³I, or ¹³²I.

15. The method of claim 10, wherein said label is an x-ray label.

16. The method of claim 10, wherein said label is a fluorescent label.

17. The method of claim 16, wherein said fluorescent label is fluorescien, lissamine, phycoerythrin, rhodamine, Cy2, Cy3, Cy3.5, Cy5, Cy5.5, Cy7, or FluorX.

18. The method of claim 10, wherein said verotoxin is the B subunit of verotoxin.

19. The method of claim 10, wherein said subject is a mammal.

20. The method of claim 19, wherein said mammal is a human.

21. The method of claim 19, wherein said mammal is a patient suffering from a cancer.

22. The method of claim 10, wherein said cell is exposed to a differentiation agent prior to administration to said subject.

23. The method of claim 10, wherein said verotoxin binding cell is a dendritic cell.

24. The method of claim 10, wherein said verotoxin binding cell is a hematopoietic progenitor cell.

25. The method of claim 10, wherein said verotoxin binding cell is administered to said subject in a pharmaceutically acceptable carrier.

26. The method of claim 10, wherein said verotoxin binding cell is administered intravenously.

27. The method of claim 10, wherein said verotoxin binding cell is administered intranodally.

28. The method of claim 11, wherein the amount of radiolabelled verotoxin administered to the subject is from about 0.1 to about 100 millicuries.

29. The method of claim 28, wherein the amount of radiolabelled verotoxin administered to the subject is from about 1 to about 10 millicuries.

30. The method of claim 29, wherein the amount of radiolabelled verotoxin administered to the subject is from about 2 to about 5 millicuries.

31. The method of claim 11, wherein said verotoxin binding cell is detected by radioscintigraphy.

32. The method of claim 10, further comprising obtaining said verotoxin binding cell from said subject.

33. A method for determining the ability of a antigen presenting cell to stimulate a T-cell response to an antigenic agent in a subject, comprising:
contacting said antigen presenting cell with a labeled verotoxin;
contacting said antigen presenting cell with an antigenic agent;
administering said antigen presenting cell to said subject; and
detecting the location of said antigen presenting cell in said subject, such that the ability of an antigen presenting cell to stimulate a T cell response is determined.

34. The method of claim 33, wherein said subject is a mammal.

35. The method of claim 34, wherein said mammal is a human.

36. The method of claim 33, wherein said labeled verotoxin is radiolabeled.

37. The method of claim 36, wherein said radio label is selected from the group consisting of ⁴³K, ⁵²Fe, ⁵⁷Co, ⁶⁷Cu, ⁶⁷Ga, ⁷⁷Br, ⁸¹Rb, ⁸¹Kr, ⁸⁷Sr, ⁹⁹Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb, and ²⁰⁶Bi.

38. The method of claim 33, wherein said antigenic agent is a verotoxin conjugate.

39. The method of claim 38, wherein said verotoxin conjugate comprises the verotoxin B subunit.

40. The method of claim 38, wherein said verotoxin conjugate comprises a bacterial, viral, or disease antigen.

41. The method of claim 38, wherein said verotoxin conjugate comprises a tumor antigen.

42. The method of claim 41, wherein said tumor antigen is from a skin, brain, ovarian, or breast tumor.

43. The method of claim 33, wherein said labeled verotoxin comprises the verotoxin B subunit.

44. The method of claim 33, wherein said antigen presenting cell is a dendritic cell.

45. The method of claim 33, wherein said antigen presenting cell is a Langerhans cell.

46. The method of claim 33, further comprising obtaining said antigen presenting cell from said subject.

47. The method of claim 33, wherein said verotoxin is labeled with a fluorescent label.

48. The method of claim 47, wherein said fluorescent label is fluorescien, lissamine, phycoerythrin, rhodamine, Cy2, Cy3, Cy3.5, Cy5, Cy5.5, Cy7, or FluorX.
